(19) United States
(12) Patent Application Publication
(10) Pub. No.: US 2022/0234928 A1
(43) Pub. Date: Jul. 28, 2022

(54) PROCESS FOR DEGRADING ORGANIC FRACTIONS IN COOLING CIRCUITS OF INDUSTRIAL PLANTS, AND COOLING CIRCUIT FOR AN INDUSTRIAL PLANT

(71) Applicant: SMS group GmbH, Düsseldorf (DE)

(72) Inventor: Angela ANTE, Hilchenbach (DE)

(73) Assignee: SMS group GmbH, Düsseldorf (DE)

(21) Appl. No.: 17/611,737

(22) PCT Filed: May 18, 2020
(86) PCT No.: PCT/EP2020/063864

§ 371 Date: Nov. 16, 2021

(30) Foreign Application Priority Data
May 16, 2019 (DE) 10 2019 207 195.3
May 12, 2020 (DE) 10 2020 002 812.8

(51) Int. Cl. C02F 3/34; A61L 2/20; A61L 2/18

(52) U.S. Cl. CPC C02F 3/343; A61L 2/202; C02F 2101/32; A61L 2/186; A61L 2/208

(57) ABSTRACT

A method for degrading organic fractions in cooling circuits in industrial plants, in particular plants in the metallurgical industry, including the following steps: adding bacteria to the cooling circuit, wherein the bacteria are suitable for degrading the organic fractions in the cooling circuit, and disinfecting the aerosol generated in a cooling tower of the cooling circuit. A cooling circuit for an industrial plant is also disclosed.

The invention relates to a method for degrading organic fractions in cooling circuits in industrial plants, in particular plants in the metallurgical industry. The invention also relates to a cooling circuit for an industrial plant, in particular for a plant in the metallurgical industry. The organic fractions in the cooling circuit of the industrial plant cause deposits, for example through increased sludge production, with the deposits having to be removed from the cooling circuit at regular intervals and disposed of separately. This increases the operating costs of the cooling circuit considerably.

It was also found that the aerosols generated in the cooling towers of cooling circuits in industrial plants can promote Legionella contamination. Therefore, the 42nd Ordinance of the Federal Immission Control Act of the Federal Republic of Germany explicitly stipulates the initiation of measures if Legionella occurs above a prescribed limit value. From the prior art, it is known, for example, to add a biocide to the cooling circuit in the event of an increased Legionella concentration, which biocide reduces the Legionella concentration to below the prescribed limit value.

It has been shown, however, that the Legionella in the sediment, deposits, and biofilms in the cooling circuit multiply and can escape again into the cooling circuit. Regular addition of biocides is therefore necessary.

Based on this prior art, the invention is based on the object of minimizing the deposits in the cooling circuit and, at the same time, ensuring a Legionella concentration below the prescribed limit values.

The object is achieved according to the invention by means of a method for the degrading of organic fractions in the cooling circuits of industrial plants, in particular plants of the metallurgical industry, comprising the following steps:
adding bacteria to the cooling circuit, the bacteria being suitable for degrading the organic fractions in the cooling circuit, and
disinfecting the aerosol generated in a cooling tower of the cooling circuit.

The first method step specifically means adding the bacteria to a coolant that circulates in the circuit. The invention is based on the knowledge that the deposits in the cooling circuit can be minimized in that the organic fractions contained in the cooling circuit are degraded. The organic fractions, in particular oils and fats, combine with solid particles in the cooling circuit and thus create the deposits. If the organic fractions are degraded, the solid particles contained in the cooling circuit are not bound, so that considerably fewer deposits result. Bacteria for degrading organic fractions in liquids are known, for example, from sewage treatment plants. However, the bacteria require certain framework conditions for the development of a biocenosis. A biocenosis within the meaning of the invention is a community of organisms in a delimited habitat (biotope), the biocenosis and the biotope together forming an ecosystem. At the same time, however, this ecosystem promotes the formation of Legionella, as the Legionella prefer framework conditions comparable to the aforementioned bacteria. The formation of a biocenosis through the added bacteria takes about 2 to 6 weeks.

The adding of biocides to the cooling circuit to reduce Legionella, which is known from the prior art, would, however, also destroy the bacterial ecosystem at the same time.

According to the invention, the Legionella that may occur, in particular Legionella pneumophila, are therefore not combated by adding a biocide, but rather by locally limited disinfecting of the aerosol generated in the cooling tower of the cooling circuit. The present invention is based on the fact that Legionella are only infectious if they get into the lungs and are pathogenic when ingested orally. An increased Legionella concentration in the cooling circuit is therefore not critical. The Legionella concentration is only critical in the area of cooling towers, where the coolant of the cooling circuit is sprayed and forms an aerosol. According to the method according to the invention, the organic fractions in the cooling circuit are degraded by the added bacteria, and any Legionella that may occur are killed off in the cooling tower of the cooling circuit. By killing off the Legionella in the cooling tower, the Legionella concentration in the entire cooling circuit is reduced at the same time, since the coolant of the cooling circuit and thus also the Legionella are inevitably circulated through the cooling tower.

The coolant used in the cooling circuit is preferably water. However, other coolants can also be used as long as they do not prevent the formation of a biocenosis by the added bacteria. The method according to the invention can also be applied to existing cooling circuits. In the start-up phase, the bacteria degrade the organic fractions in the cooling circuit and, in particular, any deposits and sediment that are present. This takes place through metabolization of the organic fractions contained in the deposits and sediment.

Since the adding of the bacteria does not require any special device, only the disinfecting of the aerosol generated in the cooling tower has to be carried out in order to use the method according to the invention in an existing cooling circuit.

According to a variant of the invention, the disinfecting of the aerosol generated in the cooling tower of the cooling circuit comprises the adding of a locally acting chemical disinfectant. The chemical disinfectant is added to the cooling circuit when it enters the cooling tower, for example. This can be done before, during, or immediately after the aerosol is generated. Examples of locally acting disinfectants are ozone, hydrogen peroxide, or peracetic acid.

According to a variant of the invention, the method comprises the step of removing excess chemical disinfectant from the coolant circuit after the cooling tower passage. Thus, after the coolant has passed through the cooling tower, any disinfectant still contained in the coolant is removed so that it cannot exert any negative influence on the added bacteria.

In a variant of the invention, steam at a temperature of 70° C. is circulated through a cooling tower of the cooling circuit to kill off the Legionella in the aerosol generated in the cooling tower. This has no negative impact on the ecosystem formed by the added bacteria.

The killing of Legionella is based on heating the Legionella to ≥70° C. Heating the coolant of the cooling circuit to such high temperatures would adversely affect the function of the cooling circuit. The use of dry heat in conjunction with a liquid coolant is also ruled out. Theoretically, however, it would be possible to kill off the Legionella by other measures, such as irradiation with UV light with at least 400 J/m², but it would have to be ensured that all surfaces are sufficiently irradiated and that the depth of penetration of the UV rays into the droplets of the aerosol is sufficient. According to the current prior art, the use of steam thus appears to be the only sensible solution. However, other measures for heating the aerosol generated in the cooling tower to at least 70° C. are not excluded according to the invention.

According to a preferred variant of the invention, bacteria with different environmental requirements are added to the cooling circuit, in particular anaerobic, anoxic, and/or aerobic bacteria. As a result, bacteria corresponding to the respective environment can propagate in the different areas of the cooling circuit, such as sedimentation tanks, clarifying tanks, filters, and the like, and form a biocenosis.

In a particularly preferred variant of the method according to the invention, nutrients are also added to the cooling circuit, in particular nutrients for the added bacteria. The added nutrients promote the formation of the biocenosis by the bacteria and also promote the long-term existence thereof.

A mixture according to the invention of added bacteria and added nutrients contains, for example, 1% bacteria and 99% nutrients.

According to an advantageous variant of the invention, the method comprises the step of adapting the ratio between added bacteria and added nutrients over time, in particular reducing the added bacteria and increasing the added nutrients over the application time of the method. For the initial formation of a biocenosis in the cooling circuit, a higher bacterial concentration is advantageous, while an already formed biocenosis can be maintained by an increased nutrient concentration without having to add larger amounts of bacteria. The concentration of added bacteria thus drops below 1% as the application time increases, while, at the same time, more than 99% nutrients are added.

According to an expedient variant of the invention, the steps of adding bacteria and/or disinfecting are repeated at regular or irregular intervals. The steps do not necessarily have to be carried out jointly or in direct succession but can also be carried out at different times and in particular at different intervals. The adding of the bacteria and possibly nutrients depend on the state of the biocenosis formed by the bacteria and is carried out according to the state of the biocenosis, while the disinfecting depends on the Legionella concentration in the coolant and only needs to be carried out if the Legionella concentration exceeds a predetermined limit value.

In a variant according to the invention, the intervals between the repetitions becomes greater as the application time of the method increases. The intervals between the adding of bacteria and/or nutrients can be increased as the method duration increases if a stable biocenosis has developed. Since the organic fractions in the cooling circuit are continuously degraded by the developed biocenosis, the deposits and sediment in the cooling circuit, which are breeding grounds for Legionella, are reduced at the same time. It can therefore be assumed that the Legionella concentration is significantly lower as the method duration increases, so that the intervals between repeated disinfection can be increased.

According to a variant of the invention, the method comprises the step of taking a sample from the cooling circuit and determining the concentration of Legionella. The sampling is expediently repeated regularly or irregularly, the intervals between the sampling preferably becoming greater as the application time of the method increases. If a Legionella concentration above a predefined limit value is determined, the disinfecting step can be carried out in order to reduce the Legionella concentration in such a way that the predefined limit value is no longer exceeded.

According to an advantageous variant, the method according to the invention is started in the winter months. In particular in the start-up phase of the method according to the invention, the intervals between repeated disinfection are shorter. Since, for example, the passage of steam at a temperature ≥70° C. through the cooling tower of the cooling circuit adversely affects the cooling performance of the cooling circuit, it is advantageous if the method according to the invention is started in the winter months with usually lower ambient temperatures, since the full cooling capacity of the cooling circuit is not required during this time.

According to a preferred variant of the invention, the cooling tower is cleaned and/or disinfected before the adding of bacteria, as a result of which any breeding grounds for Legionella that may be present are eliminated or any Legionella that may be present are killed off. Thus, only the Legionella contained in the coolant of the coolant circuit must be combated afterwards.

In an advantageous variant of the invention, the bacteria and/or nutrients are provided in the form of granules, the granules being dissolved in water before being added to the cooling circuit. Since the granules contain the bacteria and/or nutrients in concentrated form, the storage requirements are reduced. The granules are expediently dissolved in water at a temperature comparable to that of the coolant in the cooling circuit, as a result of which the propagation of the bacteria and/or nutrients in the cooling circuit is improved.

According to an expedient variant, the granules contain lyophilized bacteria. Lyophilized bacteria (freeze-dried bacteria) have a significantly longer shelf life, so that the granules can also be stored for longer periods of time.

The object is also achieved according to the invention by means of a cooling circuit for an industrial plant, in particular for plants in the metallurgical industry, comprising:
a thermal coupling to the industrial plant, and
a cooling tower for cooling the coolant in the cooling circuit,
   which is characterized in that the cooling circuit contains bacteria for degrading organic fractions and the cooling tower has a device for disinfecting the aerosol generated in the cooling tower.

The bacteria in the cooling circuit, in particular in the coolant of the cooling circuit, can form a biocenosis in one or more areas of the cooling circuit, as a result of which organic fractions are degraded in the cooling circuit. This significantly reduces the build-up of deposits and sediment. In order to prevent a Legionella concentration above a prescribed limit value at the same time, the cooling tower has the disinfection device. If the Legionella concentration rises above the limit value, the aerosol generated in the cooling tower can be disinfected, whereby the Legionella concentration in the entire cooling circuit can be reduced without affecting the one or more biocenoses formed by the bacteria.

In a variant of the invention, the device for disinfecting the aerosol generated in the cooling tower is formed as a dispensing device for a chemical disinfectant. In particular, a locally acting chemical disinfectant such as ozone, hydrogen peroxide, or peracetic acid is dispensed.

According to a variant of the invention, a device for removing excess chemical disinfectant is arranged in the cooling circuit after the cooling tower passage. This ensures that the added chemical disinfectant does not have any negative influence on the bacteria in the rest of the coolant circuit.

According to a preferred variant of the invention, the device for disinfecting the aerosol generated in the cooling tower is formed as a steam-generating unit for generating steam at a temperature ≥70° C.

In a variant of the invention, the cooling circuit comprises at least one metering device for dispensing bacteria and/or nutrients to the cooling circuit. The dispensing of the bacteria and/or nutrients can be automated by means of the metering device. In particular, the dispensing of the bacteria and/or nutrients can be adapted over the operating time of the cooling circuit and in particular automated by means of the metering device.

According to an expedient variant, the cooling circuit further comprises a sedimentation tank, a clarifying tank, and/or a filter.

According to a preferred variant according to the invention, the cooling circuit is formed to implement the method according to the invention.

The invention can be used for both direct and indirect cooling circuits. In the case of a direct cooling circuit, the cooling circuit is in direct contact with the industrial plant, while in the case of an indirect cooling circuit, a heat exchanger is arranged between the industrial plant and the cooling circuit.

In general, the invention relates to open cooling circuits with a cooling tower. In principle, however, the cooling tower can also be replaced by an evaporative cooling system or a wet separator, which, according to the invention, are also equipped with the disinfection device. The invention is also not limited to plants in the metallurgical industry, but can, in principle, also be used in other branches of industry, such as, for example, in the generation of energy in power plants.

An addition of biocide to the cooling circuit is excluded according to the invention since the biocide would destroy the biocenosis formed by the bacteria.

In the following, the invention will be explained in greater detail by means of the exemplary embodiment illustrated in the figure. The following is shown:

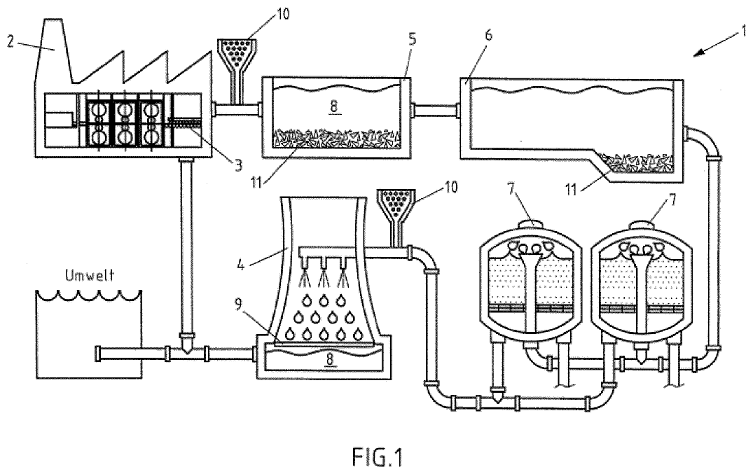

FIG. 1 a schematic view of a cooling circuit according to the invention.

The cooling circuit 1 according to the invention for an industrial plant 2 from FIG. 1 comprises a thermal coupling 3 to the industrial plant 2, a cooling tower 4, a sedimentation tank 5, a clarifying tank 6, and two filters 7. Coolant 8, preferably water, flows through the cooling circuit 1.

According to the invention, the cooling tower 4 of the cooling circuit 1 comprises a device for disinfecting the aerosol generated in the cooling tower 4. According to the exemplary embodiment from FIG. 1, the disinfection device is formed as a steam-generating unit 9 for generating steam at a temperature of 70° C. The cooling circuit 1 also comprises two metering devices 10 for dispensing bacteria and/or nutrients to the cooling circuit 1.

The heat generated in the industrial plant 2 is intended to be dissipated via the cooling circuit 1 according to the invention. For this purpose, the heat is transferred from the industrial plant 2 to the cooling circuit 1, in particular the coolant 8 located in the cooling circuit 1, via the thermal coupling 3. The heat transfer can take place directly or indirectly.

The coolant 8 is then cleaned in the sedimentation tank 5, the clarifying tank 6, and the two filters 7 before it is cooled in the cooling tower 4. The cooled coolant 8 can then be supplied back to the thermal coupling 3 or, in the case of water, for example, it can be released into the environment.

The organic fractions in the cooling circuit 1 form deposits 11, for example in the form of sludge, in particular in the sedimentation tank 5 and clarifying tank 6. These deposits have to be removed from the sedimentation tank 5 and clarifying tank 6 in a laborious manner and then disposed of separately, which is associated with correspondingly high costs.

According to the invention, it is therefore provided that bacteria are added to the cooling circuit 1, in particular the coolant 8, the bacteria being suitable for degrading the organic fractions present in the cooling circuit 1. Organic fractions are, in particular, oils and fats, which combine with solid particles in the cooling circuit 1 and thereby produce the deposits 11. The added bacteria preferably have different environmental requirements, such as anaerobic, anoxic, and/or aerobic, so that they can settle in different areas of the cooling circuit 1 and develop a biocenosis. For example, the sedimentation pit 5 is anaerobic, the clarifying tank 6 is aerobic, the filters 7 are anoxically aerobic, and the cooling tower 4 is aerobic.

According to an advantageous variant of the invention, nutrients for the added bacteria can also be added to the cooling circuit 1, in particular the coolant 8. These nutrients promote the growth of bacteria and thus the development of a corresponding biocenosis.

The ratio between added bacteria and added nutrients can be adapted over time, in particular the added bacteria are reduced and the added nutrients are increased.

The adding of bacteria and/or nutrients can be repeated at regular or irregular intervals, the intervals between the repetitions preferably increasing as the duration of the application increases.

The added bacteria significantly reduce the organic fractions in the cooling circuit 1, which leads to a significantly lower formation of deposits 11.

To ensure that the Legionella concentration in the cooling circuit 1 can be kept below a prescribed limit value, the cooling tower 4 of the cooling circuit 1 comprises the disinfection device formed as a steam-generating unit 9. By means of the steam-generating unit 9, steam, preferably water vapor, can be circulated through the cooling tower at a temperature of 70° C. As a result, the Legionella contained in the aerosol formed by the cooling tower 4 are effectively killed off, such that the Legionella concentration in the cooling circuit 1 can be reduced.

Samples are expediently taken from the cooling circuit 1, in particular from the coolant 8, at regular or irregular intervals, and the Legionella concentration is determined. If the Legionella concentration exceeds the specified limit value, steam, which is 70° C. or hotter, is circulated through the cooling tower 4 by means of the steam-generating unit 9 in order to kill off the Legionella in the generated aerosol.

As the operating time of the cooling circuit 1 increases, the intervals between the sampling can be increased, since the breeding sites for Legionella are reduced due to the fewer deposits.

The steam-generating device 1 is expediently arranged in the lower region of the cooling tower 4 so that the generated steam can rise while the aerosol generated in the cooling tower 4 falls. A good heat exchange takes place through these opposing flows.

The bacteria and/or nutrients are preferably provided in the form of granules, the granules being dissolved in water before being added to the cooling circuit 1. Since the granules contain the bacteria and/or nutrients in concentrated form, the storage requirements are reduced. The granules are expediently dissolved in water at a temperature comparable to that of the coolant 8 in the cooling circuit 1, as a result of which the propagation of the bacteria and/or nutrients in the cooling circuit 1 is improved. The granules advantageously contain lyophilized bacteria. Lyophilized bacteria (freeze-dried bacteria) have a significantly longer shelf life, so that the granules can also be stored for longer periods of time.

### LIST OF REFERENCE NUMERALS

1 Cooling circuit
2 Industrial plant
3 Thermal coupling
4 Cooling tower
5 Sedimentation tank
6 Clarifying tank
7 Filter
8 Coolant
9 Steam generator
10 Metering device
11 Deposit

1-21. (canceled)

22. A method for degrading organic fractions in cooling circuits of industrial plants, in particular of plants in the metallurgical industry, comprising:
adding bacteria to the cooling circuit, wherein the bacteria are suitable for degrading the organic fractions in the cooling circuit, and
disinfecting the aerosol generated in a cooling tower of the cooling circuit.

23. The method according to claim 22,
wherein the disinfecting comprises the adding of a locally acting chemical disinfectant.

24. The method according to claim 23,
comprising removing excess chemical disinfectant from the coolant circuit after the cooling tower passage.

25. The method according to claim 22,
comprising circulating steam at a temperature ≥70° C. through the cooling tower of the cooling circuit for heating and disinfecting the aerosol generated in the cooling tower.

26. The method according to claim 22,
wherein bacteria with different environmental requirements, in particular anaerobic, anoxic, and/or aerobic, are added to the cooling circuit.

27. The method according to claim 22,
further comprising the adding of nutrients to the cooling circuit, in particular nutrients for the added bacteria.

28. The method according to claim 27,
comprising the step of adapting the ratio between added bacteria and added nutrients over time, in particular reducing the added bacteria and increasing the added nutrients over the application time of the method.

29. The method according to claim 22,
wherein the steps of adding bacteria and/or disinfecting are repeated at regular or irregular intervals.

30. The method according to claim 26,
wherein the intervals between the repetitions becomes greater as the application time of the method increases.

31. The method according to claim 22,
comprising the step of taking a sample from the cooling circuit and determining the concentration of Legionella.

32. The method according to claim 31,
wherein the sampling is repeated regularly or irregularly, wherein the intervals between the sampling preferably become greater as the application time of the method increases.

33. The method according to claim 22,
wherein the method is started in the winter months.

34. The method according to claim 22,
wherein the bacteria and/or nutrients are provided in the form of granules, wherein the granules are dissolved in water before being added to the cooling circuit.

35. The method according to claim 34,
wherein the granules contain lyophilized bacteria.

36. A cooling circuit for an industrial plant, in particular for plants in the metallurgical industry, comprising:
a thermal coupling to the industrial plant, and
a cooling tower for cooling the coolant in the cooling circuit,
wherein
the cooling circuit contains bacteria for degrading organic fractions, and
the cooling tower has a device for disinfecting the aerosol generated in the cooling tower.

37. The cooling circuit according to claim 36,
wherein the device for disinfecting the aerosol generated in the cooling tower is formed as a dispensing device for a chemical disinfectant.

38. The cooling circuit according to claim 37,
wherein a device for removing excess chemical disinfectant is arranged in the cooling circuit after the cooling tower passage.

39. The cooling circuit according to claim 36,
wherein the device for disinfecting the aerosol generated in the cooling tower is formed as a steam-generating unit for generating steam at a temperature ≥70° C.

40. The cooling circuit according to claim 36,
further comprising at least one metering device for dispensing bacteria and/or nutrients to the cooling circuit.

41. The cooling circuit according to claim 36,
further comprising a sedimentation tank, a clarifying tank, and/or a filter.
